(19) United States
(12) Patent Application Publication
(10) Pub. No.: US 2025/0349432 A1
(43) Pub. Date: Nov. 13, 2025

(54) SYSTEM AND METHOD FOR ASSESSING RISK PREDISPOSITION TO POSTPARTUM DEPRESSION BASED ON METHYLATION MARKERS, WEARABLES, AND SURVEY DATA

(71) Applicant: Ali Mostashari, New York, NY (US)

(72) Inventors: Ali Mostashari, New York, NY (US); Mario Storga, New York, NY (US); Klemo Vladimir, New York, NY (US); Marija Majda Perisic, New York, NY (US); Raya Khanin, New York, NY (US)

(73) Assignee: LifeNome Inc., New York, NY (US)

(21) Appl. No.: 18/661,701

(22) Filed: May 12, 2024

(51) Int. Cl. G16H 50/30; C12Q 1/6876; G16H 10/20

(52) U.S. Cl. CPC G16H 50/30; C12Q 1/6876; G16H 10/20; C12Q 2600/154

(57) ABSTRACT

A method for computing predisposition risk for postpartum depression of an individual female human based at least on methylation is provided. The method comprises receiving, by a computing device, methylation data for an individual female human, the methylation data describing at least DNA methylation markers in the human. The method also comprises receiving, by the device, wearables data for the female, and receiving survey data provided by the female. The method also comprises applying, by the computing device, at least a risk predictor model builder and a risk predisposition assessment prediction algorithm to at least the received data to predict a risk predisposition to postpartum depression of the individual female. The method also comprises the computer identifying methylation markers causal to postpartum depression in the methylation data. The computer generates a personalized report describing methylation markers causal to postpartum depression, the markers identified at least in the received data.

### CROSS REFERENCE TO RELATED APPLICATIONS

None

### FIELD OF THE DISCLOSURE

The present disclosure is in the field of assessing risk predisposition to postpartum depression (PPD) during pregnancy using epigenetic markers such as methylation status of nucleotides (CpGs) in genomic DNA from biological samples. More particularly, the present disclosure provides systems and methods for identifying CpG markers causally linked to PPD, utilizing these markers to develop an accurate risk predictor of PPD using machine learning, and computing a risk predisposition assessment of a subject individual woman, in many embodiments based on a platform that integrates methylation data, self-reported data, and wearables data to update computations of the risk predisposition with a goal to identify women at early stages of elevated risk of PPD, allow for early monitoring, and personalize dietary recommendations and lifestyle changes to reduce risks of PPD.

### BACKGROUND

Postpartum depression (PPD) is a prevalent mental health condition affecting approximately 13-20% of women after childbirth. Postpartum depression (PPD) may have significant negative consequences for both the mother and the offspring (Ref. 1). PPD is characterized by a range of emotional and physical symptoms that can significantly impact a mother's ability to care for herself and her baby.

Incidence symptoms of PPD can vary from mild to severe and may manifest differently in each individual. Common symptoms include persistent feelings of sadness, hopelessness, and emptiness, a lack of interest or pleasure in activities, changes in appetite and sleep patterns, fatigue, irritability, anxiety, difficulty bonding with the baby, and thoughts of self-harm or harming the baby. PPD not only affects the mother but also influences infant development and overall family dynamics. If left untreated, it can have long-lasting effects on the mother-child relationship and the child's emotional and cognitive development (Ref 1). Early identification of high-risk women is therefore crucial to providing timely interventions and improving the quality of life for the mother, infant, and family. Susceptible women need to be identified before delivery to receive proper care measures (Ref. 2).

The etiology of PPD is multifactorial and complex. The causes of PPD are not fully understood. It is believed to be a combination of psychological factors, obstetrical factors, external stressors, and biological factors including hormonal changes according to the World Health Organization in 2001 (Ref. 1). Psychological factors include depression or anxiety experienced during pregnancy, recent stressful life events, lack of social support, previous history of depression, high levels of childcare stress, low self-esteem, difficult infant temperament, obstetric and pregnancy complications, cognitive attributions, quality of the relationship with a partner, and socioeconomic status. Obstetrical factors such as nulliparity, cesarean delivery, low breastfeeding, and parenting stress have been identified as moderate risk factors for PPD (Ref. 1).

In recent years, there has been increased progress in identifying biological predictors for PPD. These biological factors encompass anthropometric measurements, maternal age, and various biomarkers such as glucose metabolism, tryptophan, oxytocin, reproductive hormones, neurotransmitters, and neuroinflammatory biochemical factors, all of which undergo rapid changes after delivery (Ref. 3; Ref. 4).

Several studies concluded that anthropometric determinants, routinely measured by obstetricians, can be used effectively as risk markers for the severity of illness in women with postnatal depression. The waist-to-hip ratio (WHR) was found to be the most significant factor, correlated with suicidality and depression severity (Ref. 5). Both very high and very low WHR were associated with more severe symptoms, suggesting WHR as a potential marker for assessing postpartum depression risk. Other measurements, such as height, weight, and body mass index (BMI), also showed associations, although to a lesser extent.

A standard test for postpartum depression (PPD) is typically the Edinburgh Postnatal Depression Scale (EPDS). The EPDS is a self-report questionnaire that helps identify symptoms of depression in women who have recently given birth. It is widely used and has been validated for use in various cultural and linguistic settings. The EPDS provides ten questions that assess a mother's mood and emotional well-being over the previous seven days. The questions cover a range of symptoms associated with depression, such as feelings of sadness, guilt, sleep disturbances, and anxiety. Each question has a set of multiple-choice answers, and the respondent selects the response that most closely reflects her experiences.

Scores on the EPDS can range from 0 to 30, with higher scores indicating a higher likelihood of experiencing PPD. If a woman scores high on the EPDS or exhibits symptoms of postpartum depression, further assessment and evaluation should be conducted to determine the severity of the condition and develop an appropriate treatment plan. It is crucial for new mothers experiencing PPD to seek help from healthcare professionals who can provide the necessary support and treatment.

A problem with the EPDS test is that it is applied postpartum. It is a screening tool and not a diagnostic tool. A diagnosis of postpartum depression requires a comprehensive evaluation by a healthcare professional, such as a doctor or mental health specialist. It may take weeks to diagnose PPD while the health of a new mother and infant suffers.

It would therefore be beneficial to screen women who have a high risk of PPD early on. This may lead to an early referral to a psychiatrist and effective treatment, which can prevent detrimental consequences for both the mother as well as the baby. There is a significant unmet need for predictive early screening tests for PPD and developing early strategies to reduce burdens associated with it.

There is a relative lack of knowledge about the safety of standard antidepressants in the perinatal and postpartum periods. There is a clear need for more research into alternative treatments, including lifestyle changes and nutrition, such as omega-3 fatty acids, in the management of depression in the perinatal and postpartum periods.

Recent research suggests that genetics contributes to the risk of developing PPD (Ref. 6). In fact, heritability of PPD has been estimated at 54% and 44%, respectively, in twin and sibling samples. This means that about half of the variability in PPD may be explained by genetic factors (Ref. 7).

The onset of PPD within four weeks postpartum exhibits familiarity in families with major depressive disorder. These studies suggest that, while it also has its unique features, the genetic basis for PPD may partially overlap with genetic basis for other mood disorders. However, there have been relatively few studies addressing the genetic contribution to PPD compared to major depression disorder.

Earlier studies have explored a limited number of genes involved in the molecular mechanisms of PPD. It is essential to use large-scale genomics data to identify genetic variations associated with the risk of PPD, which is a complex, and likely heterogeneous disease. Therefore, it is imperative to establish a platform that can predict polygenic risk scores for PPD.

U.S. Non-Provisional patent application Ser. No. 18/447,569, filed Aug. 10, 2023, by some of the named inventors of the present disclosure and entitled “System And Method For Assessing Risk Predisposition To Postpartum Depression And Developing Personalized Lifestyle And Nutrition Plans for Use During Stages Of Preconception, Pregnancy, And Lactation/Postpartum,” addresses the development of a predictive polygenic risk score for PPD based on genetics.

Multiple studies emphasize the significance of DNA methylation in the underlying biological processes of PPD (Ref 4). A cross-species study investigating estrogen-mediated changes associated with postpartum depression identified DNA methylation profiles linked to two genes, HP1BP3 and TTC9B, associated with synaptic plasticity and estrogen signaling.

Subsequent research successfully replicated the prediction of PPD based on gene expression levels of HP1BP3 and TTC9B. These findings suggest that methylation modifications in these genes may serve as biomarkers for identifying individuals at risk for postpartum depression (Ref. 8).

Further study showed that antenatal TTC9B and HP1BP3 gene DNA methylation can predict postpartum depression (PPD) with approximately 80% accuracy suggesting the potential development of the PPD prediction model into a clinical tool for identifying pregnant women at future risk of PPD, facilitating timely intervention (Ref. 9).

Research on epigenetic modifications in the OXTR gene revealed a genotype-DNA methylation interaction in women developing PPD (Ref. 10). The study also noted a negative correlation between serum estradiol levels and DNA methylation in the OXTR gene, specifically in patients with PPD, highlighting the intricate relationship between DNA methylation, serum estradiol levels, and neuroendocrine changes in PPD.

These above-mentioned studies demonstrate the important role that DNA methylation has in the development of PPD. A significant shortcoming of the above studies is their focus on a limited number of candidate genes while PPD is a complex heterogenous disorder.

Developing a standardized approach for analyzing large-scale DNA methylation repositories is crucial to gaining further insights into the role of DNA methylation in the development of PPD. To this end, a specific embodiment of the present disclosure involves a genomics data repository that contains integrated methylation data and genetics data utilized to develop accurate risk predisposition scores of PPD.

Further, database repositories with genomics data and integrated with non-genomics repositories that contain wearables data, and survey data on PPD are available. As data repositories grow, risk score assessments based on DNA methylation data may be updated by comparing cases (pregnancies with PPD) with controls (pregnancies without PPD) using machine learning methodologies, and other computational methodologies. Risk predisposition can further be integrated into clinical practice for early identification of women with high risk.

Investment in understanding the genomic causes of postpartum depression (PPD) holds the promise of uncovering the underlying pathophysiological mechanisms, leading to potential cures or improved treatments. The critical goal is to identify women at a higher risk of PPD through genomics data and other factors, offering actionable nutritional and lifestyle recommendations to minimize risks. Ideally, this identification should take place either during the preconception stage or early in pregnancy.

Addressing the significant unmet need for accurate prediction of PPD risk, particularly based on early and modifiable biological markers such as DNA methylation markers (CpGs), requires innovative systems, methods, and devices. These advancements facilitate the early identification of women at a high risk of PPD, allowing for the exploration of novel treatments and interventions. Therefore, a key focus is on early identification using genomics data and other factors, coupled with providing actionable recommendations during the preconception stage or the first trimester of pregnancy.

### PRIOR ART

One disclosure to date addresses assessing the risk of postpartum depression (PPD) based on DNA methylation markers. The prior disclosure (WO2014071281A1) discusses a use of DNA methylation levels at loci of genes four genes (HP1BP3, TTC9B, OXTR, PABPC1L) along with white blood cell type counts, to diagnose or predict the risk of postpartum depression (PPD). A disclosed method teaches obtaining a patient sample, measuring biomarkers, including HP1BP3 and TTC9B, and assessing DNA methylation levels and white blood cell type counts. The patient is identified as likely to develop PPD based on the relative DNA methylation levels at biomarker loci in relation to the ratio of monocytes to non-monocytes. While this disclosure constitutes an attempt for an early diagnosis of PPD, the list of DNA methylation markers (CpGs) is far from exhaustive. Furthermore, it is not clear whether the measured CpGs are causally linked to GDM or the consequences of CpGs. Additionally, DNA methylation data is not integrated with wearables data, and survey/feedback data.

There are hence shortcomings regarding the assessment of PPD risks. The present disclosure provides systems and methods for risk predisposition assessment based on DNA methylation data, wearables data, and survey data to provide more accurate and early assessments, stratify population risks, and identify actionable and modifiable methylation markers.

### BRIEF DESCRIPTION OF THE FIGURES

FIG. 1 is a block diagram of a system for assessing risk predisposition to postpartum depression based on methylation markers, wearables, and survey data according to an embodiment of the present disclosure.

FIG. 2 is a block diagram of a system for assessing risk predisposition to postpartum depression based on methylation markers, wearables, and survey data according to an embodiment of the present disclosure.

FIG. 3 is a chart listing CpG markers significantly associated with postpartum depression according to an embodiment of the present disclosure.

### DETAILED DESCRIPTION

Systems and methods provided herein address deficiencies in previous implementations for assessing risk predisposition to postpartum depression (PPD) by introducing a dynamic self-learning system for deducing DNA methylation markers (CpGs) causally linked to PPD. Systems and methods provided herein further construct an accurate predictor for PPD risk utilizing a machine learning model. This model undergoes training and validation processes on constantly updated methylation data, seamlessly integrated with self-reported information and data collected from wearables.

The systems and methods disclosed herein involve the assessment of DNA methylation markers for individual women. These markers are integrated with data from wearables, as well as self-reported information obtained through surveys and feedback mechanisms. Employing machine learning methodology and perhaps other tools, systems, and methods predict the risk predisposition for PPD in subject individuals. This disclosure introduces systems and methods for the identification of DNA methylation markers causally linked to PPD.

Systems and methods disclosed herein predict the risk of PPD in individual women by leveraging methylation data, data from wearables, and self-reported information. A primary goal of this disclosure is to enhance the evaluation of PPD risk and unveil methylation markers that serve as early and modifiable biomarkers of PPD.

A platform is provided herein for collecting large amounts of heterogeneous data from individuals that may provide bases for longitudinal studies of PPD, and other pregnancy complications and pregnancy-related phenotypes. In embodiments, the platform provides personalized nutrition advice and lifestyle modifications in the stages of preconception and early pregnancy. The advice may be tailored to an individual woman's DNA methylation data, genetics data, and other considerations that may be critical to ensure the health and wellness of mothers and babies.

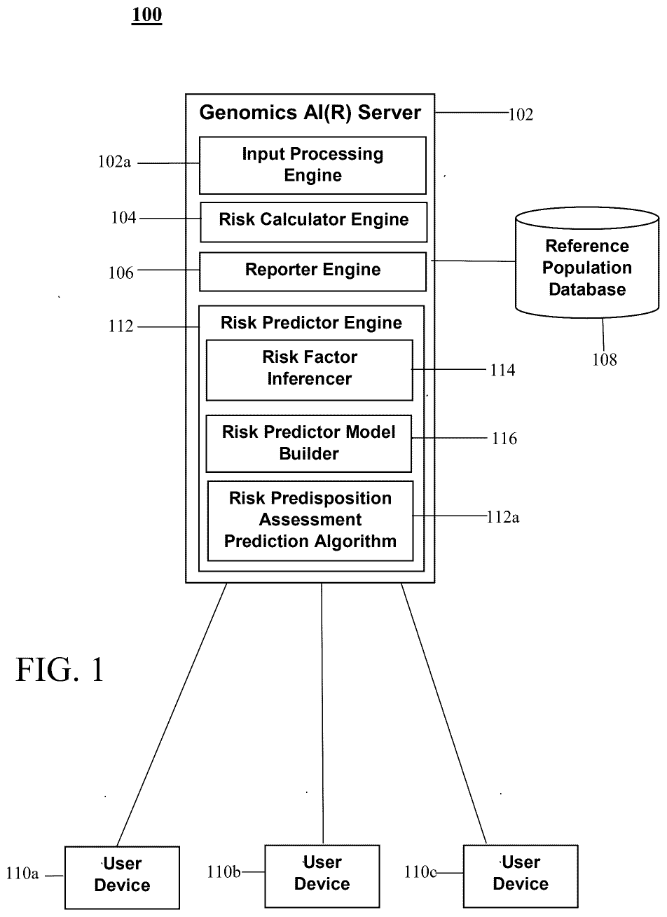

Turning to the figures, FIG. 1 illustrates the components and interactions of a system 100 for assessing risk predisposition to PPD. As depicted, the system 100 comprises a Genomics AI® server 102 that comprises an input processing engine 102a, a risk calculator engine 104, a reporter engine 106, a risk predictor engine 112, and a reference population database 108.

The system 100 also comprises a plurality of user devices 110a-c used by individuals to submit data via the input processing engine 102a to the Genomics AI® server 102 and to receive personalized reports and other data from the Genomics AI® server 102 via the reporter engine 106 and other components. The risk predictor engine 112 comprises a risk factor inferencer 114 and a risk predictor model builder 116, and a risk predisposition assessment prediction algorithm 112a. While quantity three user devices 110a-c are depicted in FIG. 1 and provided by the system 100, in embodiments more than or less than quantity three user devices 110a-c may be provided.

The Genomics AI® server 102 may be a single computer or multiple physical computers situated at one or multiple geographic locations. While the input processing engine 102a, the risk calculator engine 104, the reporter engine 106, the risk predictor engine 112, and the risk predisposition assessment prediction algorithm 112a are depicted in FIG. 1 as contained by or components of the Genomics AI® server 102 and executing on the Genomics AI® server 102, in embodiments these components shown as within the Genomics AI® server 102 in FIG. 1 may be separate hardware and/or software components executing on separate devices proximate or remote from the Genomics AI® server 102.

While referred to as engines, the input processing engine 102a, the risk calculator engine 104, the reporter engine 106, and the risk predictor engine 112 may be combinations of hardware and software applications or entirely software applications. Components described herein as modules, submodules, or devices may be physical devices, combinations of a physical device and software, or entirely software. For example, a risk factor inferencer module 114 and a risk model builder module 116 may be combinations of hardware and software or primarily software.

The Genomics AI® server 102 receives methylation data, quantities data from wearables and screening tests, and self-reported data from individuals using the user devices 110a-c. The received data is processed by the input processing device 102a of the Genomics AI® server 102 and stored in the reference population database 108.

The received data is also provided to the risk calculator engine 104 to compute a risk predisposition to PPD for an individual by applying a risk predictor model trained and validated on the reference population data in the risk predictor engine 112. The system 100 also applies algorithms comprising at least the risk predisposition assessment prediction algorithm 112a to collect and store data to assist in computing the aforementioned risk predisposition.

Based on the risk of PPD calculated by the risk calculator engine 104, the reporter engine 106 generates a personalized report for the subject individual with a predicted risk predisposition to PPD based on methylation markers. In an embodiment, the risk predisposition to PPD is based on methylation markers causal to PPD, identified in the individual's sample by the Mendelian Randomization methodology. The personalized report may further contain personalized actionable nutrition and lifestyle plans specific to the subject woman.

The personalized report may further contain a comparison of an individual's data with reference population data and contain comparisons of the individual's data at different times. The personalized report may further be utilized by the individual, or third party, for example, a healthcare professional, for recommending comprehensive monitoring and/or preventative nutrition and lifestyle programs to mitigate the risks.

Feedback collection systems may be provided that solicit and gather data from subject females and others at later times via survey questionnaires, and/or quantified data from wearables and screening tests. The gathered feedback material is provided to at least the reporter engine 106 and the reference population database 108. Additional methylation data may later be collected from individual female subjects and transmitted to the reference population database 108. Data collected at least via feedback may be utilized to build longitudinal data platforms for improving risk predisposition prediction and identifying causal methylation markers for PPD.

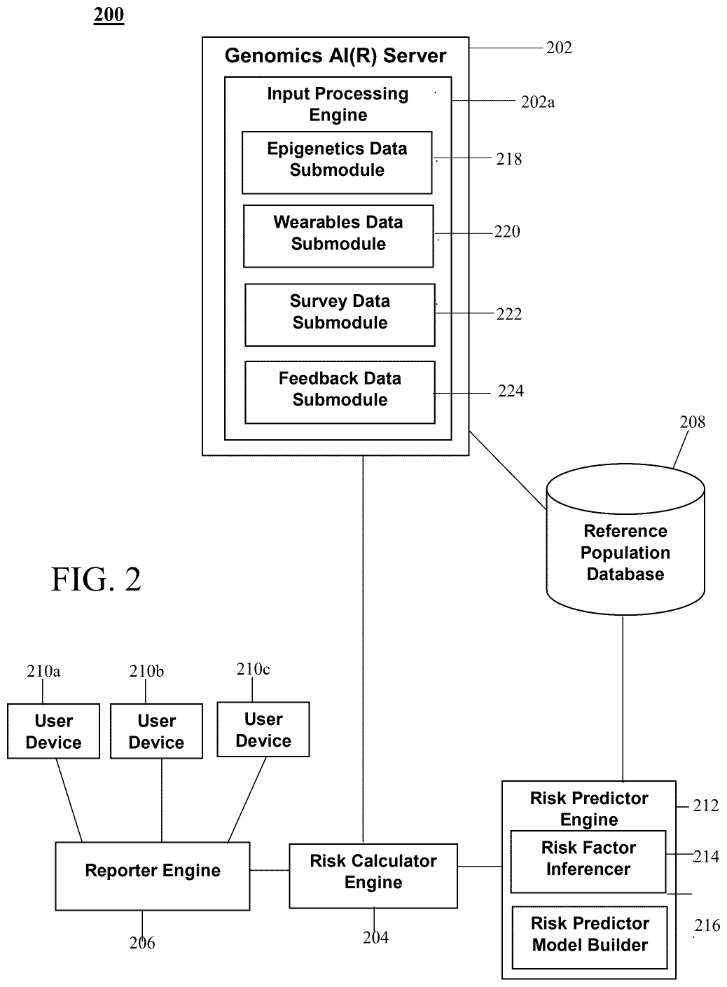

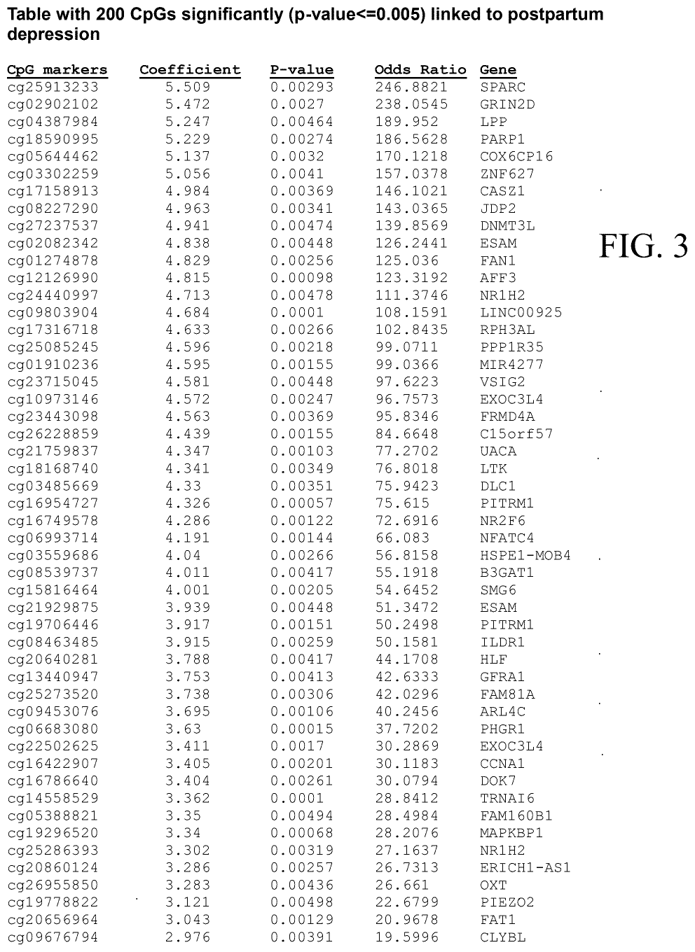

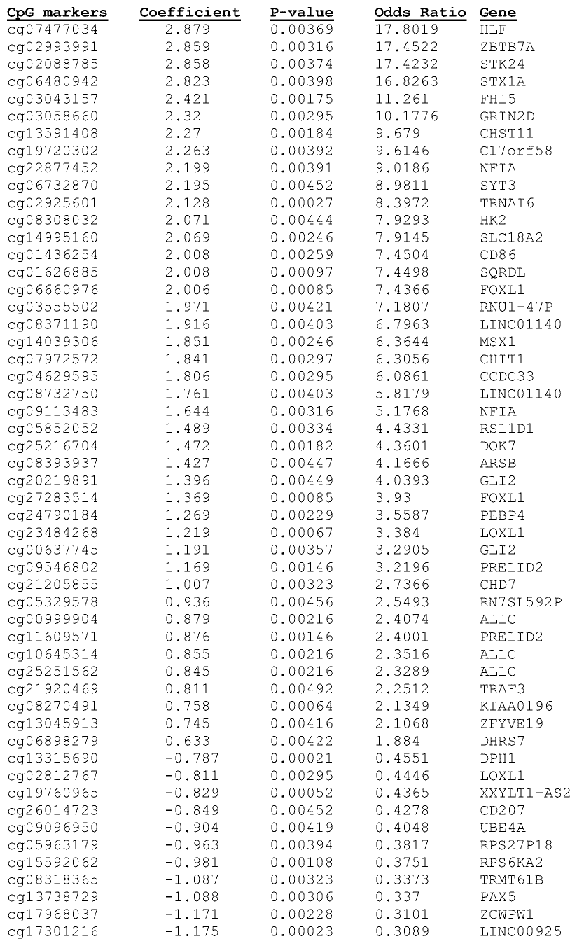

FIG. 2 is a block diagram that illustrates a variant of the structure of FIG. 1 with components of a system 200 comprising an input processing engine 202a, a reference population database 208, a risk calculator engine 204, a risk predictor engine 212, and a reporter engine 206. The input processing engine 102a receives epigenetics (methylation) data, and other information from a subject via user devices 210a-c.

The input processing engine 202a consists of four submodules: an epigenetics (methylation) data submodule 218, a wearables data submodule 220, a survey data submodule 222, and a feedback data submodule 224. In some embodiments, data input to the components of the system 200 is provided via a web, or mobile application at home, or in a professional environment at a healthcare provider.

The input processing engine 202a receives and processes methylation data from various sources via the methylation data submodule 218 which may be integrated with external information providers or databases. In some embodiments, methylation input data may be a file that contains DNA methylation markers (CpGs) uploaded by an individual, uploaded by an external genotyping or sequencing service/company using a generic or proprietary application programming interface (API), or uploaded by a third party, for example, healthcare provider, or a wellness coach. In embodiments, DNA methylation markers (CpGs) are pre-processed using appropriate bioinformatics methods directed to obtaining quantifiable results to enable further assessments.

The input processing engine 202a receives and processes data from wearables via the wearables data submodule 220. Wearables data may be generated by biosensors such as glucose monitors, wearable ECG monitors, blood pressure monitors, pulse oximeters, smartwatches with health features, temperature tracking wearable devices, sleep tracking devices, fitness tracking devices, smart rings, and smart clothing for health monitoring.

The wearables data submodule 220, which may be partially integrated with external information providers, enables input of quantified data by generic or proprietary API from sensors, wearables, and other relevant devices that report results of screening health tests or third-party expert reports, for example, physicians, healthcare providers, wellness coaches.

The input processing engine 202a receives survey data from various sources via the survey data submodule 222. Survey data may comprise chronological age, ethnicity, stage comprising preconception, pregnancy or postpartum, demographics, height, weight, activity level, diet, habits, lifestyle, medical history, geolocation, environment, and preferences. The survey data submodule 222 enables integration with self-reported questionnaires or data input by third parties.

The feedback data submodule 224 is utilized when a woman provides feedback regarding the personalized report. The feedback data submodule 224 may receive data from wearables, screening health tests, or self-reported data at stages of preconception and pregnancy. Self-reported data may contain information on adverse effects during pregnancy such as morning sickness, nausea, weight gain during pregnancy or weight loss postpartum, blood pressure, pregnancy complications, baby gestational age, baby weight, and lactation issues.

In preferred embodiments, the feedback data submodule 224 enables input of methylation data to compare methylation levels of an individual woman before and after embarking on a recommended nutrition or lifestyle plan. The feedback data submodule 224 also receives reviews, survey responses, or other feedback from the individual about specific recipes, food recommendations, and likes/dislikes. The feedback data submodule 224 may be used by the subject individual woman or a third party, for example, a healthcare professional to report adverse reactions to specific foods or recipes such as morning sickness or nausea.

Upon receipt of at least one of methylation data, wearables data, and survey data, the input processing engine 202a propagates the received data to the reference population database 208 which is a repository of at least methylation, wearables data, and survey data for a population of individuals. Material stored in the reference population database 208 is continuously updated with new entries received from individuals via the input processing engine 202a. The reference population database 208 can also be updated by bulk downloads of methylation data from multiple individuals and from public repositories of methylation data, as well as wearables data from external sources, data repositories, and third parties.

Feedback data, received from users or third parties, is propagated to the reference population database 208. After processing, using suitable data analysis tools, the feedback data is further propagated to the risk predictor engine 212 and reporter engine 206 to further improve algorithms including the risk predisposition assessment prediction algorithm 112a of the system 100, and to identify methylation markers that are either causal drivers of PPD or causal protectors from PPD.

A continuous self-learning system may thereby be set into place. For example, by analyzing, via the risk predictor engine 212, collected data in the reference population database 208, the system 200 may improve risk prediction for PPD. The system 200 may further build predictive models for other pregnancy-related complications, such as gestational diabetes, or gestational diabetes disorders.

The system may infer, by analyzing via computational algorithms and collected data, that women with specific combinations of CpG methylation markers are more likely to have more morning sickness in the first trimester if they consume specific foods. Similarly, the system may learn that specific foods and recipes help women deal with morning sickness and nausea.

The system may infer, by analyzing via computational algorithms and collected data, that specific nutritional interventions or lifestyle changes affect CpG methylation markers related to at least PPD. These nutritional and lifestyle changes are therefore utilized in the updated reports.

The reference population database 208 provides a basis for updating, via a machine learning methodology, the risk predictor engine 212. Further, the reference population database 208 may provide material that is useful for generating a personalized report by at least the reporter engine 206.

The risk predictor engine 212 comprises two modules: a risk factor inferencer 214 module and a risk predictor model builder module 216. The risk factor inferencer 214 module identifies, by applying Epigenome-Wide Mendelian Randomization (EWMR), methylation markers causal to PPD. The risk factor inferencer module 214 further validates identified methylation markers using the data from the reference population database 208.

Mendelian randomization (MR) is an established computational approach for causal inference that recapitulates the principle of a randomized clinical trial (RCT) as it utilizes genetic variants as instrumental variables. While RCTs generally consider the effect of treatment (exposure) by comparing the cases and the controls, Mendelian randomization uses genetic variants (SNPs) that are robustly associated with the exposure as instrumental variables as SNPs are randomly assigned at conception and therefore are not biased by environmental confounders. Hence, MR is used as a computational tool for investigating causal relationships between DNA methylation, as exposure, and PPD as an outcome.

In an embodiment, epigenome-wide MR (EWMR) utilizes for the outcome the summary statistics data from a genome-wide association study (GWAS) on PPD as presented in the MR-Base GWAS catalog (Ref. 11). EWMR further utilizes a publicly available dataset that has 11,165,559 SNP-CpG associations (meQTLs; P<10-14, whole blood samples) identified through GWAS from 6994 samples as exposures (Ref. 12)

In the embodiment introduced immediately above, the EWMR yields 360 CpGs that are causally linked to PPD (p<=0.01). CpGs causal to PPD can be increasing the risk of PPD (driver CpGs) or lowering the risk of PPD (protector CpGs).

The causal CpGs are mapped to causal genes. Further, the causal CpGs are mapped to causal proteins via the Epigenome-Wide Association Studies (EWAS) catalog. Functional enrichment analysis identifies that causal genes and proteins are significantly (p<0.01) enriched in multiple processes related to the development of PPD. Specifically, causal genes and proteins are enriched in processes related to brain organization and development. These processes include synapse organization, synapse assembly, structure, activity and synaptic signaling, and postsynaptic density membrane. Further, these genes and proteins are significantly enriched in processes related to axon development, axon guidance, neuron development, plasticity, and neuron death.

Causal genes and proteins are enriched in neuroinflammation and glutamatergic signaling (CAMK2D, CAMK2B, IL12A, IL12B, INSR, PRKACA, PSAT1, TNFRSF1A, TNFRSF1B, TGFBR3). This is in line with findings of disruption in neurotransmission in the underlying neurobiology of PPD, including a role for classical neurotransmitters (GABA and glutamate) and monoamines (serotonin and dopamine) (Ref. 4).

Causal genes are significantly (p-value=0.0006) enriched in oxytocin signaling pathway (RYR1, RGS2, KCNJ9, NFATC1, OXT, ITPR3, CACNG2, NFATC4). Oxytocin is known to play a critical role in the postpartum period, and multiple studies have demonstrated an inverse relationship between plasma oxytocin levels, depressive symptoms, and PPD (Ref. 10; Ref. 13).

Several pathways related to inflammatory response and immune responses, for example, cytokine signaling in the immune system, are significantly over-represented in causal genes and proteins. These findings demonstrate that CpG sites causal to PPD identified from the whole blood data may assist in elucidating biological mechanisms underlying PPD and provide clues for the discovery of drug targets.

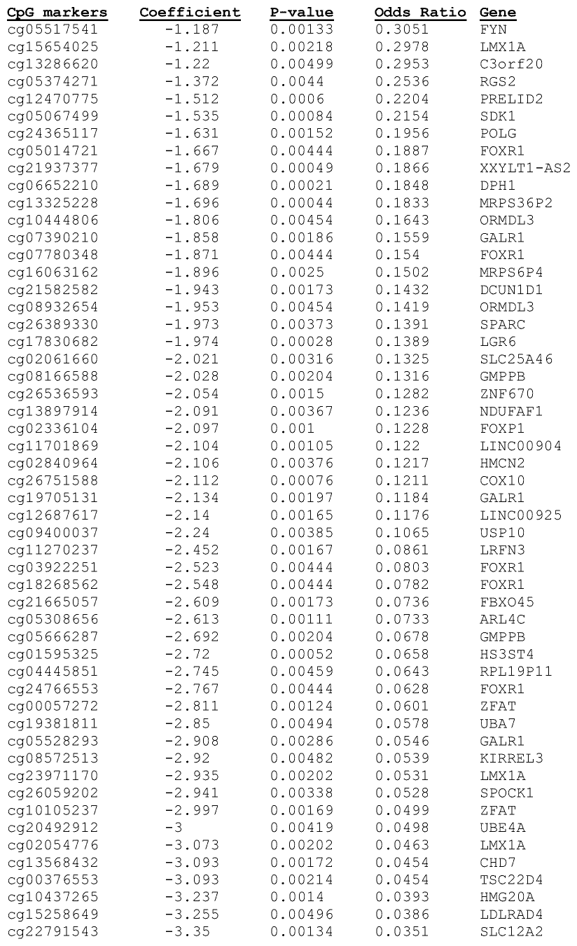

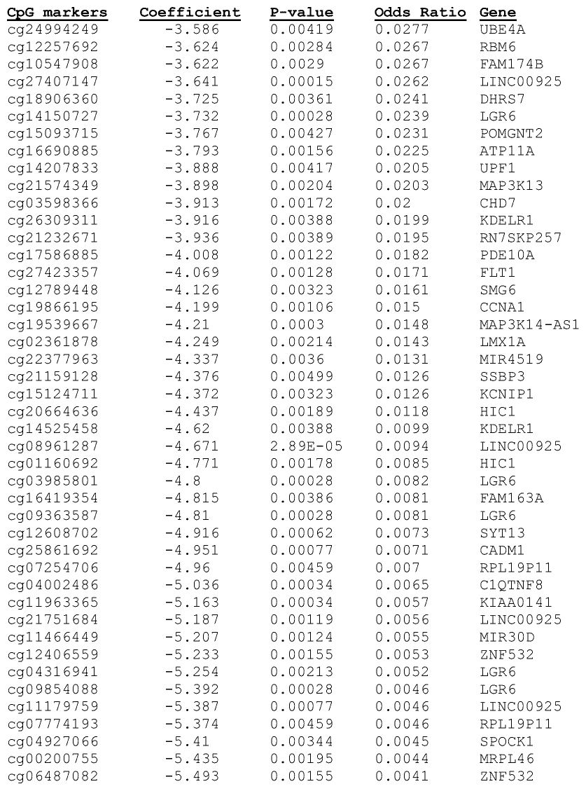

FIG. 3 is a chart listing 200 CpG markers significantly (p-value<=0.005) associated with PPD according to an embodiment of the present disclosure. There are 92 driver CpGs and 108 protector CpGs.

In other embodiments, other meQTL datasets, publicly available or proprietary, may be utilized as an exposure in EWMR. An example is the GoDMC meQTL dataset that contains SNP-CpG associations for 420,509 CpG sites identified in whole blood samples from 27,750 subjects. In other embodiments, pregnancy complications such as gestational diabetes mellitus, gestational hypertension, preeclampsia, adverse cardiac events, and pregnancy-related phenotypes such as morning sickness, nausea, weight gain during pregnancy or weight loss postpartum, baby gestational age, baby weight, and lactation issues can be used as outcomes in the EWMR analyses to infer methylation markers causal to pregnancy and postpartum complications.

In certain embodiments, Mendelian Randomization takes the form of a two-sample Mendelian Randomization utilizing linear regression. Alternatively, in specific instances, it adopts a three-sample Mendelian Randomization approach. Furthermore, in other embodiments, Mendelian Randomization employs non-linear models to depict the relationship between exposure and outcome. Those skilled in the art understand that various models, both linear and nonlinear, can be constructed between exposures and outcomes to deduce causal methylation markers.

The risk prediction model builder 116 develops, via a supervised machine learning methodology, a predictive model for PPD using data from the reference population database 108. In a preferred embodiment, CpGs causal to PPD are utilized as input features. The risk prediction model builder 116 may perform the computations of predictive risk prediction models using at least one algorithm that may be proprietary and/or developed by a third-party source and following best practices of machine learning.

The risk predictor model builder 116 develops a methylation risk score (MRS) predicting the current risk of PPD. The MRS model is built, using a machine learning methodology, on a training dataset that consists of human blood samples of cases (women with PPD) and controls (women who did not have PPD). Each sample has methylation markers measured by external genotyping or sequencing service. In a preferred embodiment, MRS is a risk-weighted linear sum of methylation levels at several CpG sites (Ref. 14).

Validation of risk predictors may be carried out on an independent dataset of biological samples. In preferred embodiments, both the training and validation datasets incorporate proprietary data. Publicly available datasets are integrated with proprietary data, subject to pre-processing and normalization through bioinformatics methods. The datasets are then partitioned into training and validation subsets to ensure at least age balance.

In embodiments, the risk predictor model 116 is learned using an elastic net model using, as input features, CpG markers causal to PPD as identified by EWMR in the risk inference module. In other embodiments, the risk predictor is learned using an elastic net model using, as features, CpG markers that are significantly associated with PPD, and other relevant markers extracted from wearables data, screening tests, or survey data.

In a specific embodiment, the elastic net model includes the CpG feature-specific penalty factor informed by the causality rank that is based on at least one of the causal effect sizes and p-values from EWMR analyses or a quantitative measure computed by a bioinformatics tool (e.g. colocalization probability). In other embodiments, the risk predictor is learned by another supervised machine learning algorithm, wherein CpG features are ranked by taking the causality rank into account. In another embodiment, CpG features are ranked by a quantitative measure based on correlations with hypertension and other markers extracted from wearables data, screening tests, or survey data.

The risk calculator engine 104 is a computing device that receives methylation data from an individual via the input processing engine 102a. It further receives causal methylation markers and the risk predictor model from the risk predictor engine 112. The risk calculator engine 104 further identifies causal methylation markers in the individual's methylation data and calculates risk predisposition to PPD of the individual using a risk predictor model.

The reporter engine 106 receives causal methylation markers and risk predisposition to PPD for an individual from the risk calculator engine 104 and generates a personalized report informing individuals about their risk predisposition to PPD, methylation markers that contribute to the risk, and methylation markers that protect from the risk. In other embodiments, specific foods and recipes can be identified, by computational analyses, as reversing the effect of specific damaging methylation markers or improving protective methylation markers.

Feedback provided by individuals is done via user devices 110a-c that collect data and responses from individuals on the provided, by the system, personalized reports. Feedback data may comprise methylation data collected from individuals at different time points. Feedback data may further comprise wearables data and data from survey questionnaires. Feedback responses may comprise results from questionnaires on comprehension of information provided by personalized reports.

Feedback responses may comprise liking/disliking recommendations of foods and recipes. Feedback data may then be transmitted to the reference population database 108 by using the feedback data submodule 224 of the system 200, and, after processing, be further transmitted to the risk predictor engine 112 to improve risk prediction algorithms. A continuous self-learning system may thereby be set in place. Feedback responses may be transmitted to the reporter engine 106 to improve personalized reports.

In some embodiments, user devices 110a-c may be mobile computing devices such as a smartphone or a tablet computing device. In some embodiments, user devices 110a-c may be a desktop computing device or a laptop computing device. In some embodiments, the user devices 110a-c may include more than one computing device, such as a user computing device 110a-c configured to provide a user interface and one or more server computing devices configured to provide computational functionality. In such embodiments, the user computing device 110a-c and one or more server computing devices may communicate via any suitable communication technology or technologies, such as a wired technology (including but not limited to Ethernet, USB, or the Internet) or a wireless technology (including but not limited to WiFi, WiMAX, 3G, 4G, LTE, or Bluetooth).

In an embodiment, a method for computing predisposition risk for postpartum depression of an individual female human based at least on methylation is provided. The method comprises receiving, by a computing device, methylation data for an individual female human, the methylation data describing at least DNA methylation markers in the female human. The method also comprises receiving, by the computing device, wearables data for the female. The method also comprises receiving, by the computing device, survey data provided by the female. The method also comprises applying, by the computing device, at least a risk predictor model builder and a risk predisposition assessment prediction algorithm to at least the received data to predict a risk predisposition to postpartum depression of the individual female. The method also comprises the computer identifying methylation markers causal to postpartum depression in the methylation data.

The method also comprises the computer generating a personalized report for the individual, the report describing risk factors from wearables data, the report describing the computed predisposition risk assessment based at least on methylation markers, and the report further describing methylation markers causal to postpartum depression, the markers identified at least in the received data.

The method also comprises the computer utilizing data collected at least via feedback to build a longitudinal data platform for improving risk predisposition prediction and identifying causal methylation markers for PPD. The method also comprises the computing device transmitting the computed predisposition risk and the methylation data, the wearables data, and the survey data for the female to a data repository that stores at least reference population methylation data, wearables data, and survey data for a plurality of women.

The risk predisposition predictor model is trained and validated on reference population data stored in the reference population database. Wearables data of the individual female are provided by biosensors comprising wearable ECG monitors, blood pressure monitors, pulse oximeters, smartwatches with health features, temperature-tracking wearables, sleep trackers, fitness trackers, smart rings, and smart clothing for health monitoring. Risk factors of postpartum depression are extracted, via a machine learning (AI) classifier, from the wearables data, wherein the classifier is one of a proprietary, an open-source, and a third-party algorithm utilized via an application programming interface (API).

In another embodiment, a system for continual improvement of risk predisposition assessment to postpartum depression based at least on methylation data is provided. The system comprises a computer and application executing thereon that receives epigenetics data containing at least DNA methylation markers describing an individual female. The system also receives wearables data describing the individual female. The system also receives feedback data and survey data comprising at least a chronological age of the individual female. The system also computes a risk predisposition assessment to postpartum depression of the individual female based on the data. The system also propagates the received data and the predicted risk predisposition assessment to postpartum depression to a reference population storage.

The system uses the received data and previously stored data to improve a risk predisposition assessment prediction algorithm, the algorithm selectively used in computing their risk predisposition assessment. The feedback data is further propagated to a risk predisposition assessment predictor engine and a reporter engine to improve the risk predisposition assessment prediction algorithm and identify methylation markers that are either causal drivers of postpartum depression, including preeclampsia, or causal protector methylation markers of postpartum depression.

DNA methylation markers (CpGs) are pre-processed using bioinformatics methods directed to obtaining quantifiable results to enable further assessments. The system enables input of methylation data to compare risk predisposition to postpartum depression of individuals before and after recommended nutritional and lifestyle programs. he system builds predictive models for risk predisposition assessments to pregnancy-related or postpartum-related phenotypes comprising at least one of postpartum depression, gestational hypertension disorders, preeclampsia, gestational diabetes mellitus, cardiac complications, morning sickness, and nausea.

In yet another embodiment, a method for using methylation markers associated with pregnancy-related phenotypes is provided. The method comprises a computer applying Epigenome-Wide Mendelian Randomization (EWMR) to received data describing at least one individual female. The method also comprises the computer identifying, via the applied EWMR, methylation markers (CpGs) causal to at least one pregnancy-related phenotype. The method also comprises the computer utilizing epigenome-wide methylation (meQTL) data as exposure. The method also comprises the computer validating the identified methylation markers.

The method also comprises the computer validating the markers using data from a reference population database. The method also comprises the computer applying the EWMR to utilize summary statistics from genome-wide association studies for pregnancy-related phenotypes as outcomes.

The method also comprises the computer observing and measuring risk factors from at least one of wearables data, survey data, and feedback data. Epigenome-wide methylation data (meQTL) contain SNP-CpG associations detected in a biological sample comprising at least one of whole blood, and saliva. Methylation markers (CpGs) associated with at least one pregnancy-related or postpartum-related phenotype are identified by one of the correlative analyses and generalized linear regression from reference population data and wherein pregnancy-related phenotype data are at least one of observable and measurable and are extracted from at least one of pregnancy-related phenotype data, wearables data, survey data, and feedback data.

Steps of systems and methods of some embodiments provided herein may be as follows:
1. The system 100 receives an individual woman's epigenetic data (DNA methylation markers, CpGs).
2. The system 100 adds the individual's methylation data to a population's methylation data and compares the woman's methylation data to reference population data comprising at least the population's methylation data
3. The system 100 further receives wearables data of the woman, via a wearable device, or sensor.
4. The system 100 adds the individual's wearables data to population wearables data, compares the individual's wearables data to the reference population wearables data, and integrates population wearables data with population methylation data.
5. The system 100 further receives survey data from the individual woman, wherein the data can be self-reported or collected by a healthcare provider. Survey data includes at least an individual's age, and it may further include information on general health, diet, and lifestyle.
6. The system 100 adds the individual woman's survey data to the reference population survey data, compares the individual woman's survey data to the reference population survey data, and integrates population methylation data with population wearables data and population survey data.
7. The system 100 collects longitudinal data that includes methylation data, wearables data, and survey data from a plurality of individuals measured at various time intervals. Survey data may include feedback on food and recipe recommendations, including liking/disliking, subjective assessments, and adverse effects. Survey data may be self-reported or reported by a third party.
8. The system 100 propagates the individual woman's longitudinal data to storage with population data.
9. The system 100 computes risk predisposition to PPD for the individual woman by utilizing a risk predictor model using a plurality of methylation markers identified in the methylation data of the individual woman.
10. The system generates a personalized report that contains the individual woman's predicted risk predisposition to PPD, related methylation markers, measurements from wearables data, and relevant markers from screening tests.
11. The system relies on a reporting and feedback module to send and receive the material.

### REFERENCES

1. Gibson-Smith et al. Association of food groups with depression and anxiety disorders. Eur J Nutr. 2020 Mar;59(2):767-778. doi: 10.1007/s00394-019-01943-4. Epub 2019 Apr 3. PMID: 30945032; PMCID: PMC7058560.
2. Bhatia. Maternal risk markers of postnatal depression. J Postgrad Med. 2020 Jan-Mar;66(1):7-8. doi: 10.4103/jpgm.JPGM_350_19. PMID: 31898595; PMCID: PMC6970328.
3. Bloch et al. Endocrine factors in the etiology of postpartum depression. Compr Psychiatry. 2003 May-Jun;44(3):234-46. doi: 10.1016/S0010-440X (03) 00034-8. PMID: 12764712.
4. Payne and Maguire. Pathophysiological mechanisms implicated in postpartum depression. Front Neuroendocrinol. 2019 Jan;52:165-180. doi: 10.1016/j.yfrne.2018.12.001. Epub 2018 Dec. 12. PMID: 30552910; PMCID: PMC6370514.
5. Nayak and Nachane. Maternal anthropometric determinants as risk markers of suicidality and severity of illness in women with postnatal depression. J Postgrad Med. 2020 Jan-Mar;66(1):11-16. doi: 10.4103/jpgm.JPGM_541_18. PMID: 31898598; PMCID: PMC6970329.
6. Payne. Genetic basis for postpartum depression. In: Biomarkers of Postpartum Psychiatric Disorders. London: Elsevier. (2020) p. 15-34. doi: 10.1016/B978-0-12-815508-0.00002-3.
7. Viktorin et al. Heritability of perinatal depression and genetic overlap with non-perinatal depression. Am J Psychiatry. (2016) 173:158-65. doi: 10.1176/appi.ajp.2015.15010085.
8. Guintivano et al. Antenatal prediction of postpartum depression with blood DNA methylation biomarkers. Mol Psychiatry. 2014 May;19(5):560-7. doi: 10.1038/mp.2013.62. Epub 2013 May 21. Erratum in: Mol Psychiatry. 2014 May; 19(5):633. PMID: 23689534; PMCID: PMC7039252.
9. Payne et al. DNA methylation biomarkers prospectively predict both antenatal and postpartum depression. Psychiatry Res. 2020 Mar;285:112711. doi: 10.1016/j.psychres.2019.112711. Epub 2019 Nov. 27. PMID: 31843207; PMCID: PMC7702696.
10. Bell et al. Interaction between oxytocin receptor DNA methylation and genotype is associated with risk of postpartum depression in women without depression in pregnancy. Front Genet. 2015 Jul 21;6:243. doi: 10.3389/fgene.2015.00243. PMID: 26257770; PMCID: PMC4508577.
11. Hemani et al. The MR-Base platform supports systematic causal inference across the human phenome. eLife. 2018;7:e34408. doi: 10.7554/eLife.34408. PMID: 29846171; PMCID: PMC5976434.
12. Hawe et al. Genetic variation influencing DNA methylation provides insights into molecular mechanisms regulating genomic function. Nat Genet. 2022 Jan;54(1):18-29. doi: 10.1038/s41588-021-00969-x. Epub 2022 Jan. 3. PMID: 34980917.
13. Thul et al. Oxytocin and postpartum depression: A systematic review. Psychoneuroendocrinology. 2020 Oct;120:104793. doi: 10.1016/j.psyneuen.2020.104793. Epub 2020 Jul. 6. PMID: 32683141; PMCID: PMC7526479.
14. Thompson et al. Methylation risk scores are associated with a collection of phenotypes within electronic health record systems. NPJ Genom Med. 2022 Aug 25;7(1):50. doi: 10.1038/s41525-022-00320-1. PMID: 36008412; PMCID: PMC9411568.

What is claimed is:

1. A method for computing predisposition risk for postpartum depression of an individual female human based at least on methylation, comprising:
receiving, by a computing device, methylation data for an individual female human, the methylation data describing at least DNA methylation markers in the female human;
receiving, by the computing device, wearables data for the female;
receiving, by the computing device, survey data provided by the female; and
applying, by the computing device, at least a risk predictor model builder and a risk predisposition assessment prediction algorithm to at least the received data to predict a risk predisposition to postpartum depression of the individual female.

2. The method of claim 1, further comprising the computer identifying methylation markers causal to postpartum depression in the methylation data.

3. The method of claim 1, further comprising the computer generating a personalized report for the individual, the report describing risk factors from wearables data, the report describing the computed predisposition risk assessment based at least on methylation markers, and the report further describing methylation markers causal to postpartum depression, the markers identified at least in the received data.

4. The method of claim 3, further comprising utilizing data collected at least via feedback to build a longitudinal data platform for improving risk predisposition prediction and identifying causal methylation markers for PPD.

5. The method of claim 1, further comprising the computing device transmitting the computed predisposition risk and the methylation data, the wearables data, and the survey data for the female to a data repository that stores at least reference population methylation data, wearables data, and survey data for a plurality of women.

6. The method of claim 1, wherein the risk predisposition predictor model is trained and validated on reference population data stored in the reference population database.

7. The method of claim 1, wherein wearables data of the individual female is provided by biosensors comprising wearable ECG monitors, blood pressure monitors, pulse oximeters, smartwatches with health features, temperature-tracking wearables, sleep trackers, fitness trackers, smart rings, and smart clothing for health monitoring.

8. The method of claim 7, wherein risk factors of postpartum depression are extracted, via a machine learning (AI) classifier, from the wearables data, wherein the classifier is one of a proprietary, an open-source, and a third-party algorithm utilized via an application programming interface (API).

9. A system for continual improvement of risk predisposition assessment to postpartum depression based at least on methylation data, comprising:
a computer and application executing thereon that:
receives epigenetics data containing at least DNA methylation markers describing an individual female,
receives wearables data describing the individual female,
receives feedback data and survey data comprising at least a chronological age of the individual female,
computes a risk predisposition assessment to postpartum depression of the individual female based on the data, and
propagates the received data and the predicted risk predisposition assessment to postpartum depression to a reference population storage.

10. The system of claim 9, wherein the system uses the received data and previously stored data to improve a risk predisposition assessment prediction algorithm, the algorithm selectively used in computing their risk predisposition assessment.

11. The system of claim 9, wherein the feedback data is further propagated to a risk predisposition assessment predictor engine and a reporter engine to improve the risk predisposition assessment prediction algorithm and identify methylation markers that are either causal drivers of postpartum depression, including preeclampsia, or causal protector methylation markers of postpartum depression.

12. The system of claim 9, wherein DNA methylation markers (CpGs) are pre-processed using bioinformatics methods directed to obtaining quantifiable results to enable further assessments.

13. The system of claim 9, wherein the system enables input of methylation data to compare risk predisposition to postpartum depression of individuals before and after recommended nutritional and lifestyle programs.

14. The system of claim 9, wherein the system builds predictive models for risk predisposition assessments to pregnancy-related or postpartum-related phenotypes comprising at least one of postpartum depression, gestational hypertension disorders, preeclampsia, gestational diabetes mellitus, cardiac complications, morning sickness, and nausea.

15. A method for using methylation markers associated with pregnancy-related phenotypes, comprising:
a computer applying Epigenome-Wide Mendelian Randomization (EWMR) to received data describing at least one individual female;
the computer identifying, via the applied EWMR, methylation markers (CpGs) causal to at least one pregnancy-related phenotype;
the computer utilizing epigenome-wide methylation (meQTL) data as exposure; and
the computer validating the identified methylation markers.

16. The method of claim 15, further comprising the computer validating the markers using data from a reference population database.

17. The method of claim 15, further comprising the computer applying the EWMR to utilize summary statistics from genome-wide association studies for pregnancy-related phenotypes as outcomes.

18. The method of claim 15, further comprising the computer observing and measuring risk factors from at least one of wearables data, survey data, and feedback data.

19. The method of claim 15, wherein epigenome-wide methylation data (meQTL) contain SNP-CpG associations detected in a biological sample comprising at least one of whole blood, and saliva.

20. The method of claim 15, wherein methylation markers (CpGs) associated with at least one pregnancy-related or postpartum-related phenotype are identified by one of the correlative analyses and generalized linear regression from reference population data and wherein pregnancy-related phenotype data are at least one of observable and measurable and are extracted from at least one of pregnancy-related phenotype data, wearables data, survey data, and feedback data.
